(19) United States
(12) Patent Application Publication
(10) Pub. No.: US 2024/0317976 A1
(43) Pub. Date: Sep. 26, 2024

(54) MAGNETIC HYDROGEL WITH A TERNARY MAGNETIC COMPOSITE AND METHOD FOR FABRICATING THE SAME

(71) Applicants: Versitech Limited, Hong Kong (HK); Advanced Biomedical Instrumentation Centre Limited, Hong Kong (HK)

(72) Inventors: Lizhi XU, Changsha (CN); Hengjia ZHU, Hangzhou (CN); Zuochen WANG, Lanzhou (CN)

(21) Appl. No.: 18/610,295

(22) Filed: Mar. 20, 2024

Related U.S. Application Data
(60) Provisional application No. 63/491,959, filed on Mar. 24, 2023.

(51) Int. Cl. C08K 9/04; C08K 3/22; C08K 7/02; C08L 29/04; H01F 1/44; A61B 34/00

(52) U.S. Cl. CPC C08K 9/04; C08K 3/22; C08K 7/02; C08L 29/04; H01F 1/445; A61B 34/73; C08K 2003/2275; C08K 2201/003; C08K 2201/01; C08K 2201/011; C08L 2205/16

(57) ABSTRACT

The present invention provides a class of magnetic hydrogels with tissue-mimetic mechanical properties and photothermal welding/healing capability. In these hydrogels, a hybrid network involving aramid nanofibers, functionalized Fe₃O₄ nanoparticles, and polyvinyl alcohol (PVA) is accomplished by a stepwise assembly of the functional components. The engineered interactions between nanoscale constituents enable facile materials processing and confer a combination of excellent mechanical properties, magnetism, water content and porosity.

### Cross-Reference to Related Applications

The present application claims the priorities from the U.S. provisional patent application Ser. No. 63/491,959 filed Mar. 24, 2023, and the disclosure of which is incorporated herein by reference in its entirety.

### FIELD OF THE INVENTION

The present invention generally relates to material science. More particularly, it relates to a high-strength magnetic hydrogel, which can be further applied to technical fields such as in implantable soft robots, drug delivery system, human-machine interactions, etc.

### BACKGROUND OF THE INVENTION

The burgeoning field of soft robotics calls for advanced materials that can be actuated in response to external stimuli. Among the variety of soft active materials, magnetic hydrogels are particularly useful for biomedical applications since they are water-rich and can be remotely controlled with the bio-compatible magnetic field. They could be implemented in advanced tools for drug delivery, tissue engineering platforms, human-machine interactions, and many other technologies. However, existing magnetic hydrogels exhibit limited mechanical strength and manufacturability, which restrict their practical applications. Indeed, the required inorganic magnetic components often disrupt the structural properties of hydrogels that highly rely on the porous network of polymers. On the other hand, the complex synthesis schemes for mechanically strong hydrogels might not be compatible with functional design of the composites or fabrication of hybrid device structures.

In biological soft tissues, such as skin, blood vessels, ligaments, etc., an excellent combination of mechanical properties, functionality and water retention was accomplished with the hybrid nanofiber networks involving collagen and other soft biopolymers. They provide a robust structural basis for various physiological functionalization, allowing for the formation of heterogeneous organs that operate under dynamic mechanical deformation. Emulating these features of natural soft tissues would enable a versatile materials platform for the construction of reliable functional structures for advanced bio-integrated systems. However, it remains difficult due to the limited synthetic building blocks.

Recently emerged nanocomposites based on aramid nanofibers (ANFs) provide a new route for the engineering of magnetic hydrogels that could address these challenges. ANFs self-assemble into 3D networks that exhibit microstructural properties similar to those of natural collagen fibers, providing a biomimetic framework for the construction of various soft composites. The polar groups on ANFs allow interactions with other organic materials, facilitating the formation of functional hydrogels that retain the high structural robustness arising from the nanofiber skeleton.

Nevertheless, incorporation of magnetic components in the ANF network is still difficult. For instance, in-situ synthesis of magnetic nanostructures highly relies on surface chemistry, wherein the functional groups determine nanoparticle nucleation and growth. Due to the chemical stability of para-aramid (poly (p-phenylene terephthalamide)) (PPTA) chains, chemical modification of ANFs requires complex and cumbersome procedures that might not be compatible with magnetic components. Physical deposition methods, such as vacuum assisted filtration and matrix infiltration, require diffusion of functional building blocks in aqueous or vapor phase. In the presence of the significant interactions between magnetic particles, these processes may cause severe aggregation or non-uniform distribution of the particles, leading to compromised functionality or structural failure by stress concentration. Therefore, developing strategies for reliable incorporation of magnetic components into ANF-based composites hydrogels becomes essential.

### SUMMARY OF THE INVENTION

It is an objective of the present invention to provide a facile fabrication strategy for ANF-based magnetic hydrogels with high mechanical strength and excellent manufacturability.

In accordance with a first aspect of the present invention, it provides a high-strength magnetic hydrogel having a ternary magnetic composite. The formulation of the magnetic hydrogel includes a plurality of aramid nanofibers, one or more magnetic components capable of dispersing in an organic solvent for at least 24 hours and a polymer matrix. The organic solvent contains at least one hydrogen bonding acceptor. Hydroxyl groups and amine groups on the one or more magnetic components spontaneous packs with the plurality of aramid nanofibers via hydrogen bonding, and the one or more magnetic components are organized around the three-dimensional fibrous network formed by the plurality of aramid nanofibers.

In accordance with one embodiment of the present invention, the plurality of aramid nanofibers includes an aramid polymer selected from Kevlar®, Twaron® Nomex®, or a combination thereof.

In accordance with one embodiment of the present invention, the one or more magnetic components comprise Fe₃O₄—NH₂ nanoparticles or gold nanoparticles. The one or more magnetic components have a particle size ranging from 15 nm to 50 nm.

In accordance with another embodiment of the present invention, the concentration of the one or more magnetic components ranges from 5 wt % to 50 wt %.

When the concentration of the one or more magnetic components reaches to at least 20%, the magnetic hydrogel exhibits a similar mechanical robustness compared to a binary magnetic composite.

In accordance with another embodiment of the present invention, the polymer matrix includes polyvinyl alcohol (PVA), polyethylene glycol (PEG).

In accordance with another embodiment of the present invention, the organic solvent includes dimethyl sulfoxide (DMSO), dimethylformamide (DMF), acetone, or a combination thereof.

In accordance with a second aspect of the present invention, the magnetic hydrogel is further processed by near-infrared induced welding with one or more non-magnetic hydrogel to foam a heterogeneous hydrogel structure.

In accordance with a third aspect of the present invention, it provides a method for preparing a high-strength magnetic hydrogel, including mixing a colloidal suspension and an aramid nanofiber suspension in a solvent to form a first mixture; adding 2-10 mL of polyvinyl alcohol solution in the solvent to the first mixture to obtain a ternary mixture; and immersing the ternary mixture in water to form the magnetic hydrogel. The colloidal suspension comprises one or more magnetic components. The ternary mixture is in a form of a viscous and moldable fluid without gelation.

In accordance with another embodiment of the present invention, the step of immersing the ternary mixture in water to form the magnetic hydrogel further includes performing solvent exchange. The strong hydrogen bonding acceptors are exchanged by a weaker hydrogen bond acceptor, such as water.

In accordance with another embodiment of the present invention, the plurality of aramid nanofibers can be an aramid polymer.

In accordance with another embodiment of the present invention, the one or more magnetic components include Fe₃O₄—NH₂ nanoparticles or gold nanoparticles, with a particle size ranging from 15 nm to 50 nm.

In accordance with another embodiment of the present invention, the solvent includes dimethyl sulfoxide (DMSO), dimethylformamide (DMF), acetone, or a combination thereof.

In accordance with another embodiment of the present invention, the ratio between the colloidal suspension and the aramid nanofiber suspension is 1:20 to 1:1.

In accordance with another embodiment of the present invention, the ratio between the first mixture and the polyvinyl alcohol solution is 1:10 to 1:2.

The present invention provides a facile fabrication strategy for aramid nanofibers (ANF)-based magnetic hydrogels with high mechanical strength and excellent manufacturability. Its high welding efficiency provides promising potential for customizable functionality of devices.

The magnetic hydrogels of the present invention have the following advantages: (1) aramid based composite magnetic hydrogel allowing for robust and durable products; and (2) simple fabrication process allowing high customizability for integrated electronic systems.

### BRIEF DESCRIPTION OF THE DRAWINGS

Embodiments of the present invention are described in more detail hereinafter with reference to the drawings, in which:

FIG. 1 depicts a schematic diagram of formation process of the hierarchical structures;

FIG. 2 shows images of the molding process of ANF-Fe₃O₄-PVA composite hydrogels;

FIG. 3A shows a TEM image showing Fe₃O₄—NH₂ nanoparticles, where the aggregation was induced by drop casting method using DI water. Scale bar: 200 nm.

FIG. 3B depicts an electron diffraction pattern of the functionalized Fe₃O₄ nanoparticles;

FIG. 4 shows images showing dispersion stability of Fe₃O₄—COOH and Fe₃O₄—NH₂ in water;

FIG. 5 depicts (upper) poor solvation of the Fe₃O₄—NH₂ in water, with severe aggregation of nanoparticles, and (bottom) good solvation of Fe₃O₄—NH₂ in DMSO, with well dispersion of nanoparticles more than 24 hours;

FIG. 6 shows SEM images showing the morphology of (left) ANF-PVA hydrogel and (right) ANF-Fe₃O₄-PVA composite hydrogel after critical point drying, where the nanoparticles are embedded on the surface of the nanofibers. Scale bar: 1 μm;

FIG. 7 shows images of fabricated ANF-PVA and ANF-Fe₃O₄-PVA composite hydrogels, and NIR welded hydrogel samples, and programed magnetic response of the samples;

FIG. 8A shows images of a sample of ANF-Fe₃O₄-PVA-1 magnetic hydrogel with 0% (left) and 66.6% (right) tensile strains. FIG. 8B shows images of ANF-Fe₃O₄-PVA-1 magnetic hydrogel without (left) and with (right) a compressive load of 20 N (2 kg);

FIG. 9A depicts compressive curves for ANF-PVA, and ANF-Fe₃O₄-PVA magnetic hydrogels with four concentrations. FIG. 9B depicts stress-strain curves for ANF-PVA, and ANF-Fe₃O₄-PVA magnetic hydrogels with four concentrations;

FIG. 10 depicts cyclic tensile and compressive tests of ANF-Fe₃O₄-PVA-1. The stress-strain curve indicates that the stress-strain curve is dependent on the maximum loading;

FIG. 11 depicts tensile and compressive moduli of various ANF-Fe₃O₄-PVA magnetic composite hydrogels;

FIG. 12 shows SEM images showing the morphology of ANF-Fe₃O₄-PVA hydrogel samples with increasing content of Fe₃O₄ (from ANF-Fe₃O₄-PVA-1 to ANF-Fe₃O₄-PVA-4). The circle highlights the excess magnetic nanoparticles. Scale bar: 1 μm;

FIG. 13 shows Infrared images showing the temperature change of the hydrogel from 28.93° C. to 470.25° C. in 2 minutes under the illumination of a NIR laser;

FIG. 14 shows images showing the separated hydrogel thin films (left) and thick stripes (right) can be welded together by the NIR laser;

FIG. 15 depicts stress-strain curves for the welded hydrogels;

FIG. 16 depicts welded hydrogels under ambient temperature and NIR laser irradiation, and corresponding length-temperature plot showing temperature distribution on an array of functionalized Fe₃O₄ nanoparticles under NIR laser, where the center part of the array shows the highest temperature due to the coupling effect;

FIG. 17 shows SEM images at different welding stages showing that the interfaces are experiencing merging processes, and finally fused together. Scale bar: 20 μm;

FIG. 18 shows SEM images of side and cross-section view of the burned ANF-Fe₃O₄ aerogel, where the ANFs transformed into beads;

FIG. 19A depicts images of a stretching process applied on welded hydrogel samples with thin film. FIG. 19B depicts images of a stretching process applied on welded hydrogel samples with thick film. FIG. 19C depicts heterojunction of ANF-PVA and ANF-Fe₃O₄-PVA composite hydrogel;

FIG. 20A depicts tensile stress between pristine ANF-Fe₃O₄-PVA-1 and same hydrogels experienced cut-welding process. FIG. 20B depicts strain at break between pristine ANF-Fe₃O₄-PVA-1 and same hydrogels experienced cut-welding process. The thickness of thin films and thick strips are 80 μm and 250 μm, respectively;

FIG. 21 shows images showing a LED powered through a conductive hydrogel sample before and after the cutting-healing process;

FIG. 22 depicts resistance change of ANF-Au-PVA composite hydrogel as a strain sensor under tensile deformation;

FIG. 23 depicts a schematic diagram of various recycling processes of aramid materials, including those based on chemical stripping, mechanical fibrillation, and NIR-aided welding;

FIG. 24 depicts the welding process of a flower-like heterostructure fabricated from ANF-Fe₃O₄-PVA and ANF-PVA hydrogels;

FIGS. 25A-25E depict reversible actuation of hydrogel structures with pure ANF-Fe₃O₄-PVA (FIG. 25A) and heterostructures with one (FIG. 25B), two (FIG. 25C), or three (FIG. 25D) pieces of magnetic hydrogel patches, as well as a complex flower-like structures (FIG. 25E). The actuation was performed under an inhomogeneous 300 mT magnetic field applied perpendicular to the plane. Scale bar: 2 cm;

FIG. 26 depicts fluorescent images (top) and cell viability histogram (bottom) of fibroblasts cultured on ANF-Fe₃O₄-PVA magnetic composite hydrogel, measured on day 1, day 3, and day 5 after cell seeding. Scale bar: 100 μm; and

FIG. 27 depicts fluorescence microscope images of live and dead fibroblasts cultured on ANF-Fe₃O₄-PVA magnetic composite hydrogels, measured on day 1, day 3, and day 5 after cell seeding. Scale bars: 100 μm.

### DETAILED DESCRIPTION

In the following description, high-strength magnetic hydrogels and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

Hydrogels capable of transforming in response to magnetic field hold great promise for applications in soft actuators and biomedical robots. However, achieving high mechanical strength and good manufacturability simultaneously in magnetic hydrogels remains challenging.

Hence, inspired by natural load-bearing soft tissues, the present invention develops an effective strategy for integrating magnetic components into aramid nanofiber (ANF)-based composite hydrogels reliably.

In a first aspect, the present invention provides a high-strength magnetic hydrogel having a ternary magnetic composite. The formulation of the hydrogel includes a plurality of aramid nanofibers, one or more magnetic components capable of dispersing in an organic solvent for at least 24 hours, and a polymer matrix.

Hydroxyl groups and amine groups on the one or more magnetic components spontaneous packs with the plurality of aramid nanofibers via hydrogen bonding, and the one or more magnetic components are organized around the three-dimensional fibrous network formed by the plurality of aramid nanofibers.

The components of aramid nanofibers primarily contain aramid polymers, typically poly (p-phenylene terephthalamide) (PPTA) or its derivatives. Examples of the derivatives may include meta-aramid polymers, such as poly(m-phenylene isophthalamide) (PMIA); a aramid polymer copolymerized with other monomers. For example, copolymers of PPTA with other aromatic monomers. These polymers are characterized by their exceptionally high strength, stiffness, heat resistance, and chemical stability. The aramid nanofibers are typically produced through polymerization of aramid precursors and subsequent spinning into fibers. These fibers are then manipulated, often through stretching or other means, to achieve nanoscale dimensions.

In one embodiment, the one or more magnetic components may include Fe₃O₄—NH₂ nanoparticles or gold nanoparticles.

In one embodiment, the polymer matrix may include polyvinyl alcohol, polyethylene glycol.

These hydrogels exhibit magnetic properties due to the incorporation of magnetic particles, and they possess the structural and mechanical properties conferred by the aramid nanofibers.

Furthermore, the present invention also provides a method for preparing a high-strength magnetic hydrogel, including mixing a colloidal suspension and an aramid nanofiber suspension in a solvent to form a first mixture; adding an equal volume of polyvinyl alcohol solution in the solvent to the first mixture to obtain a ternary mixture; and immersing the ternary mixture in water to form the high-strength magnetic hydrogel.

The step of immersing the ternary mixture in water to form the magnetic hydrogel further comprises performing solvent exchange.

The ratio between the colloidal suspension and the aramid nanofiber suspension is 1:20 to 1:1.

The ratio between the first mixture and the polyvinyl alcohol solution is 1:10 to 1:2.

The hybrid network involving aramid nanofibers, Fe₃O₄ nanoparticles with amine groups, and polyvinyl alcohol (PVA) is achieved through a stepwise assembly of the functional components. Firstly, aramid nanofibers serve as the framework, providing structural support and mechanical strength. Subsequently, Fe₃O₄—NH₂ nanoparticles are introduced into the network. These nanoparticles possess magnetic properties, imparting magnetic responsiveness to the material. Finally, PVA, as a water-soluble polymer, enhances the material's water solubility and stability through interactions with other components. Through this stepwise assembly approach, a composite network structure is formed, exhibiting tissue-mimetic mechanical properties and photothermal welding/healing capability.

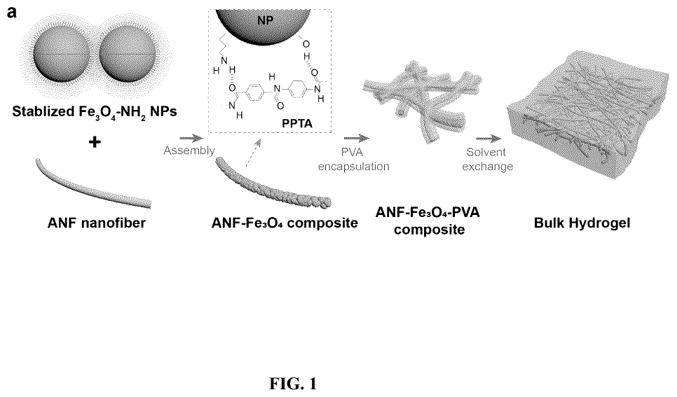

Turning to FIG. 1, a high-strength magnetic hydrogel is made by the following steps: assembling Fe₃O₄—NH₂ nanoparticles with aramid nanofibers to form an ANF-Fe₃O₄ composite materials; adding an equal volume of polyvinyl alcohol solution in the DMSO to the ANF-Fe₃O₄ composite materials to form ANF-Fe₃O₄-PVA composite materials; and performing solvent exchange to generate the high-strength magnetic hydrogel.

The following examples illustrate the present invention and are not intended to limit the same.

### EXAMPLE

### Example 1

Synthesis of Fe₃O₄ nanoparticles with amine groups on the surface

To match the diameter of the aramid nanofibers, the functionalized Fe₃O₄ nanoparticles with 20-30 nm in diameter were synthesized. Briefly, 0.6 g of FeCl₃ as ferro source was dissolved with 6.5 g of 1,6-hexanediamine and 2.0 g anhydrous sodium acetate in 30 mL glycol by 50° C. vigorously stirring and sonification. The fully dissolved, transparent solution was transferred into a Teflon-lined autoclave and put into oven at 198° C. for 6 h. The magnetite nanoparticles were then washed by ethanol and DI water via magnet collection and dispersion process (2 to 3 times) to remove the residue reactant, and finally dispersed in DI water.

Due to the strong dipolar interactions of magnetic particles, the particles tended to form clusters and even aggregate. The particles were then washed by DMSO for three times through magnet induced precipitation and redispersion process, forming stable suspension prior to hydrogel fabrication. TEM (FEI Tecnai G2 20 Scanning TEM) was employed to characterize the nanoparticles.

### Example 2

Fabrication of ANF-Fe₃O₄-PVA hydrogels or ANF-Au-PVA hydrogels

Turning now to the drawings in detail, FIG. 1, to fabricate composite hydrogels with desirable properties in bulk, a stepwise assembly strategy was employed. Preparation of the composite hydrogel started with intermixing the colloidal suspensions of Fe₃O₄—NH₂ nanoparticles formed in the Example 1 and a plurality of aramid nanofibers in dimethyl sulfoxide (DMSO). The hydroxyl and amine groups on the magnetite nanoparticles allowed spontaneous packing with the aramid nanofibers via hydrogen bonding. The DMSO suspension of ANF-Fe₃O₄ was then mixed with DMSO solution of PVA, which further enhanced stretchability and toughness of the nanofiber networks.

In one embodiment, the ANF suspension (2 wt %) in DMSO was prepared and mixed with the Fe₃O₄—NH₂ DMSO suspension from Example 1. The mixing ratio could be varied by changing the concertation of nanoparticles, while the volume of the suspension kept constant. The mixture was stirred vigorously for 2 minutes to ensure adequate assembly with the Fe₃O₄—NH₂ nanoparticles through hydrogen bonding. Then, a 10 wt % of PVA DMSO solution with an equal volume to ANF suspension was added, followed by another 2 min of stirring.

This process was highly water sensitive and should be performed under an inert environment, such as nitrogen gas (in a glove box). Any water absorption might induce the microscale ANF gelation and lead to micro phase separation, which was detrimental for their mechanical performance. The mixture was either coated as film or cast in customized molds. The shaped mixture was then immersed in DI water to generate ANF-Fe₃O₄-PVA hydrogel.

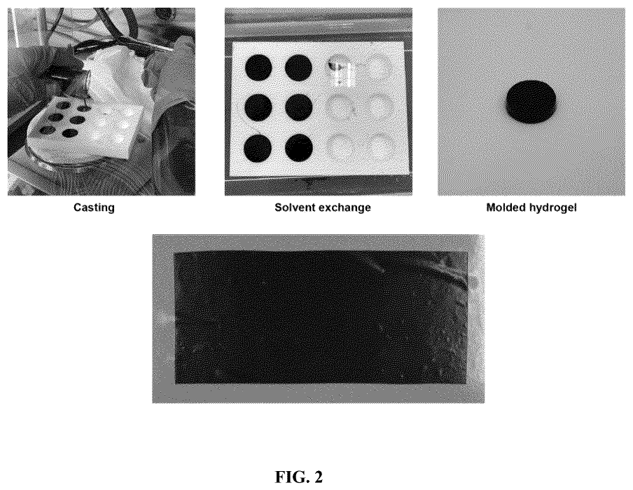

The ternary mixture appeared a viscous and moldable fluid (FIG. 2), since DMSO was a strong hydrogen bonding acceptor and prevent gelation that was unwanted at this stage. After the mixing steps, a solid hydrogel could be obtained when the mixture was immersed in water, wherein the DMSO was exchanged by a weaker hydrogen bond acceptor, such as water.

Fabrication procedures for ANF-PVA and ANF-Au-PVA hydrogels were the same, where ANF-PVA hydrogels did not require addition of any nanoparticles, and ANF-Au-PVA hydrogels added Au nanoparticles instead of Fe₃O₄ nanoparticles. For SEM (Hitachi S4800 FEG SEM) characterization, supercritical CO2 drying was employed to remove water from the hydrogel matrix without further damage.

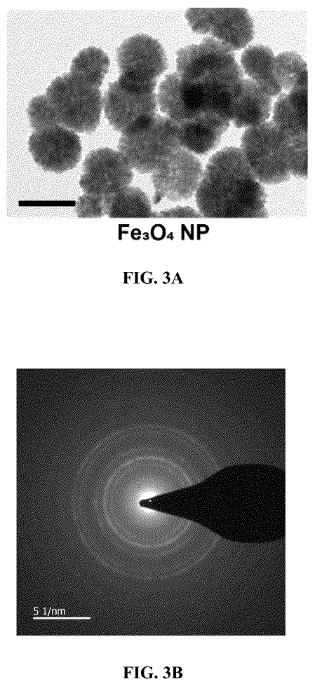

To match the length scale of the nanofibers, functionalized Fe₃O₄ nanoparticles with diameters of about 25 nm were synthesized. The nanoparticles were surface functionalized with alkyl chains terminated with amine and hydroxyl groups (FIG. 3A). The Fe₃O₄ lattices were further verified by TEM ring diffraction pattern (FIG. 3B), which was consistent with theoretical calculations based on the reported lattice parameters (Table 1).

TABLE 1
Electron diffraction pattern comparison with theoretical
calculation of d-spacing of the Fe₃O₄ lattice.

| | |
|---|---|
| | Measured diffraction |
| Theoretical calculation | pattern |

| d- | | | | | d- |
|---|---|---|---|---|---|
| spacing | | | | | Spacing |
| [nm] | h | k | l | Spot# | [nm] |
| 0.48514 | 1 | 1 | 1 | 1 | 0.5008 |
| 0.29709 | 2 | 2 | 0 | 2 | 0.2986 |
| 0.25336 | 3 | 1 | 1 | 3 | 0.2549 |
| 0.24257 | 2 | 2 | 2 | 4 | 0.2116 |
| 0.21007 | 4 | 0 | 0 | 5 | 0.1726 |
| 0.19277 | 3 | 3 | 1 | 6 | 0.1644 |
| 0.17152 | 4 | 2 | 2 | 7 | 0.1506 |
| 0.16171 | 5 | 1 | 1 | 8 | 0.1279 |
| 0.14854 | 4 | 4 | 0 | | |
| 0.14203 | 5 | 3 | 1 | | |
| 0.13286 | 6 | 2 | 0 | | |
| 0.12814 | 5 | 3 | 3 | | |
| 0.12668 | 6 | 2 | 2 | | |
| 0.12128 | 4 | 4 | 4 | | |
| 0.11766 | 5 | 5 | 1 | | |
| 0.11229 | 6 | 4 | 2 | | |
| 0.10939 | 7 | 3 | 1 | | |

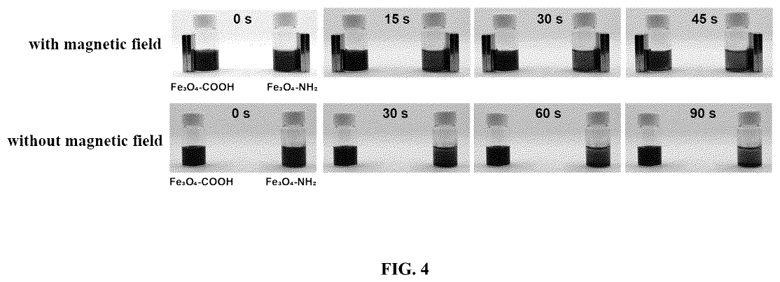

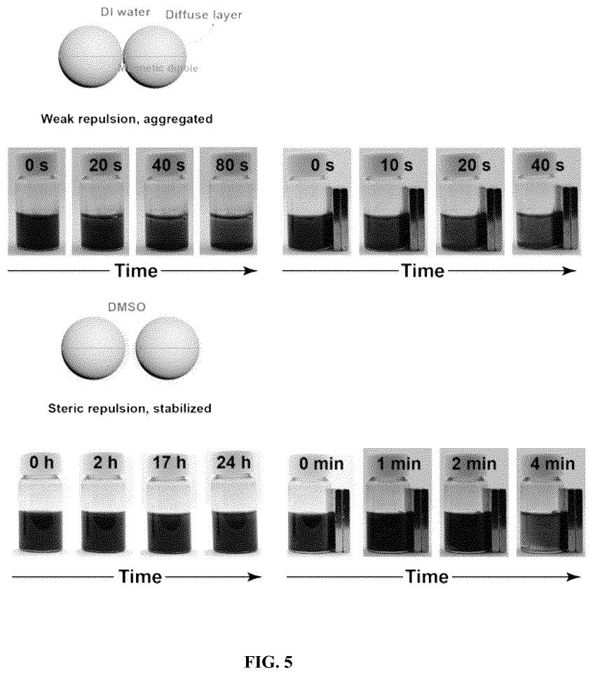

During synthesis of Fe₃O₄—NH₂ nanoparticles, 1,6-hexanediamine was used as the modulator, which helped to control the size of the particles and produces sufficient alkyl chains and amine groups on their surfaces. In deionized (DI) water, the surface alkyl chains closely packed on the surface of Fe₃O₄—NH₂ nanoparticles due to their poor solubility in water. The dissociation of amine and hydroxyl groups was thus limited, producing weak electrostatic repulsion between nanoparticles. The nanoparticles aggregated due to dominated dipolar and van der Waals attractions and settled to the bottom of glass vials within seconds (FIG. 4), which contrasted with highly charged Fe₃O₄—COOH particles that only contain surface hydroxyl groups (FIG. 5). However, in an organic solvent (e.g., DMSO), the alkyl chains were highly dissolved and solvated. The steric repulsion of Fe₃O₄—NH₂ nanoparticles counteracted the dipolar attraction or van der Waals interactions, forming stable colloidal suspension (FIG. 1 and FIG. 4).

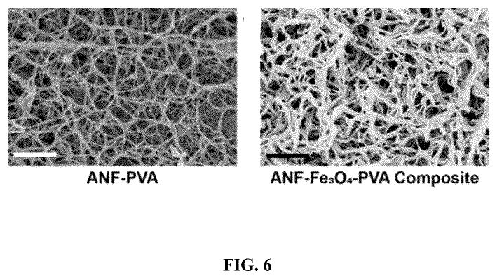

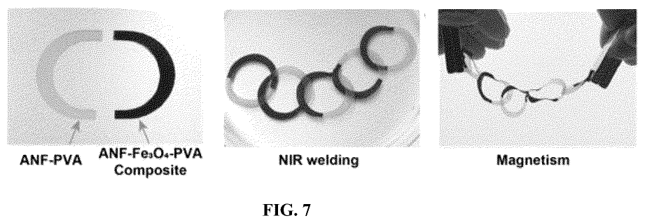

The SEM images showed the microscale morphology of the ANF-Fe₃O₄-PVA hydrogels. The Fe₃O₄—NH₂ nanoparticles attached to the nanofibers without significant aggregation or disruption of the 3D interconnected network (FIG. 6). It was conceivable that the nanofiber network provided an excellent template to guide the assembly of Fe₃O₄—NH₂ nanoparticles and acted as a robust structural framework to bear mechanical loads. In addition, due to the inherent magnetic and photothermal properties of Fe₃O₄—NH₂ nanoparticles, the ANF-Fe₃O₄-PVA and ANF-PVA hydrogels could be further processed with NIR welding to form hybrid structures with designed magnetic responses (FIG. 7).

### Example 3

Mechanical properties of ANF-Fe₃O₄-PVA hydrogels

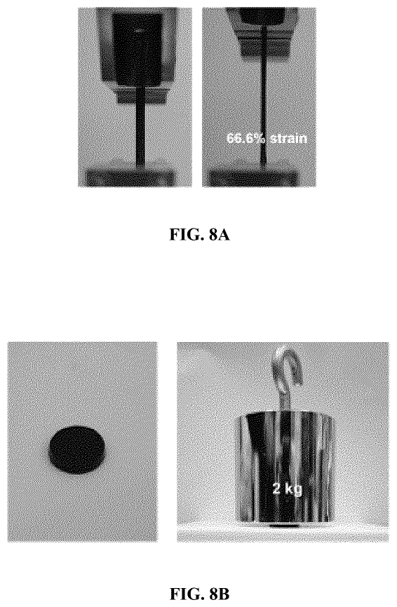

Due to the hybrid nanofiber network, ANF-Fe₃O₄-PVA hydrogels were mechanically strong and stretchable (FIG. 8A-8B). To quantitatively analyze their mechanical properties, tensile and compression tests on pure ANF-PVA hydrogels and ANF-Fe₃O₄-PVA hydrogels with various concentrations of nanoparticles were performed.

The mechanical tests were performed under ambient condition using a universal testing machine (Zwick Roell, German). For tensile tests, thick (250 μm) and thin (80 μm) rectangular strips with 5 mm in width and 15 mm in length were prepared using laser cutting machine and tested under strain rate at 100%/min. For compression test, cylindrical samples with 10 mm diameter and 3 mm in thickness were prepared by pouring and degassing mixed solution into Teflon molds. The compression tests were performed under strain rate at 100%/min. For healed samples, the tensile tests were performed under strain rate at 100%/min.

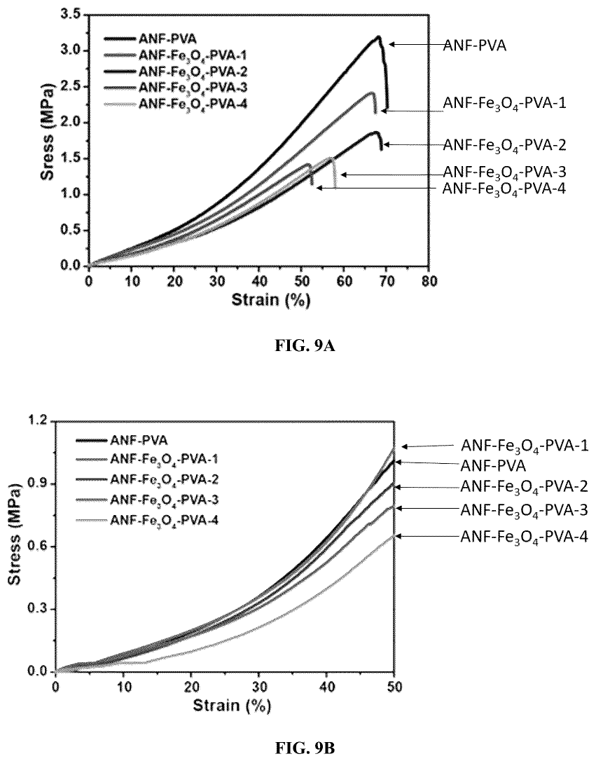

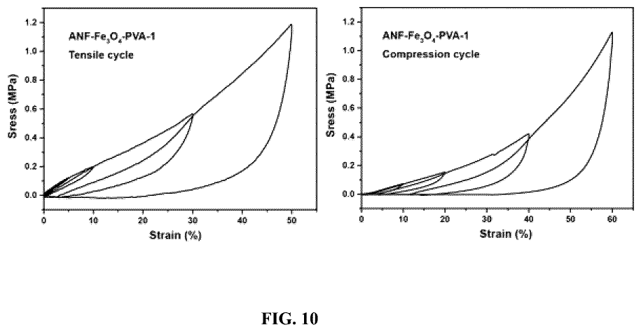

Referring to FIG. 9A-9B, the magnetic composite hydrogels exhibited high strength (1.32-2.4 MPa) and stretchability (53.3%-66.6%) similar to those of pure ANF-PVA hydrogels. However, the Fe₃O₄ nanoparticles functionalized with amine or hydroxyl groups might have a slight interference with the hydrogen bonding between ANFs and PVA, which played a significant role in the mechanical properties of the hydrogels. With lower nanoparticle concentrations (7.3 wt % or 13.7 wt % of dried samples), the fracture strains of ANF-Fe₃O₄-PVA hydrogels were similar to that of pure ANF-PVA hydrogel (FIG. 9A). The energy dissipation characteristics of these ANF-Fe₃O₄-PVA hydrogels were also similar to those of pure ANF-PVA (FIG. 10).

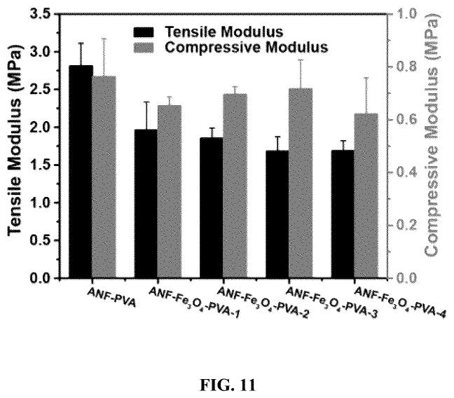

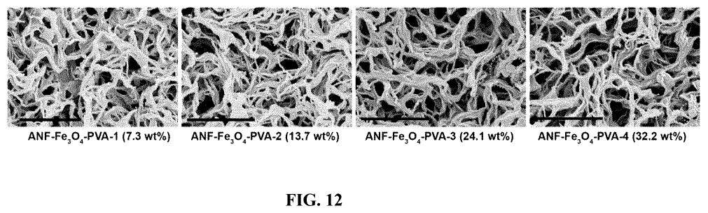

Samples with higher nanoparticle concentrations (24.1 wt % or 32.2 wt %) exhibit decreased in tensile strength and facture strain (FIG. 11). From SEM examination, all of the samples retained the 3D nanofiber network originating from the ANF framework (FIG. 12), although excess particles could be found in samples with high Fe₃O₄ loading. Further optimization of the magnetic responses and mechanical properties of the composite hydrogels would be based on specific device applications.

The compressive behaviors of ANF-Fe₃O₄-PVA hydrogels were less dependent on the loading of nanoparticles. Samples loaded with Fe₃O₄—NH₂ nanoparticles retained similar compressive responses as the pure ANF-PVA hydrogels (FIG. 9B). Different from the tensile responses that mostly arise from the nanofiber network, the compression resistance of the hydrogels was also related to the trapping of interstitial water flow that was less dependent of nanoparticle loading. Overall, the mechanical robustness of the ANF-Fe₃O₄-PVA hydrogels was sufficient for device applications even with high loading of Fe₃O₄—NH₂ nanoparticles.

### Example 4

Near-infrared (NIR) induced welding of ANF-Fe₃O₄-PVA hydrogel

Welding and fusing of functional hydrogels are important for the fabrication of hybrid devices or repair of damaged structures. Existing processing methods for soft materials, including thermal annealing, solvent treatment, or chemical cross-linking, lack the capability in spatially selective welding. In addition, given the good solvent stability and low thermal conductivity of ANFs combined with the water-rich feature of the matrix, these traditional methods are incapable of welding ANF-based composite hydrogels.

On the other hand, incorporating photothermally actively materials, such as gold (Au) nanoparticles or organic dyes dispersed in the composites, might enable selective welding via laser irradiation. However, the use of noble nanoparticles as functional components dramatically increases the cost and complexity of the synthesis process, whereas the organic dye molecules cannot sustain the high temperature required to soften the polymer composites. Interestingly, the photothermal properties of Fe₃O₄—NH₂ nanoparticles embedded in the PVA-rich composite hydrogels may enable a convenient method for selective welding through NIR laser irradiation.

In the present invention, the photothermal property of Fe₃O₄—NH₂ nanoparticles organized around the nanofiber network allowed near infrared (NIR) welding of the hydrogels, providing a versatile means to fabricate heterogeneous structures with custom designs. Complex modes of magnetic actuation were made possible with the manufactured heterogeneous hydrogel structures, suggesting opportunities for further applications in implantable soft robots, drug delivery system, human-machine interactions, and other technologies.

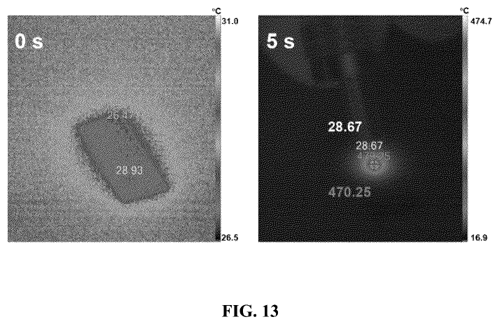

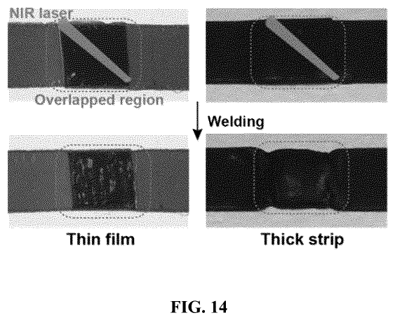

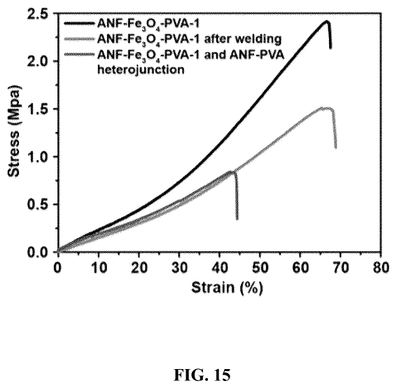

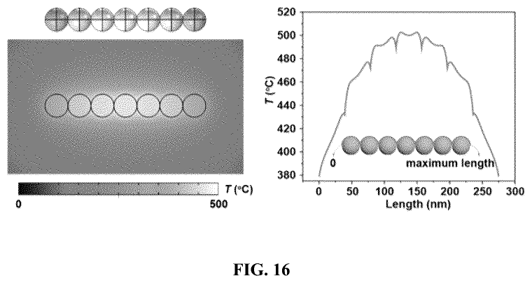

Prior to NIR welding, the ANF-Fe₃O₄-PVA hydrogel samples were fabricated in bulk by doctor blade coating method. After solvent exchange process, the hydrogel samples were laser cut into desired shapes. The NIR welding was realized by the photothermal effect of Fe₃O₄—NH₂ nanoparticles. Two pieces of ANF-Fe₃O₄-PVA hydrogel films with the same width were overlapped along their long axis, followed by the NIR laser illumination (785-nm, Shanghai Laser & Optics Century, IRM785RMA-300FC). The NIR power (100-300 mW) and welding duration (1-5 min) relied on the thickness of films and the demand of mechanical strength at the joint, both enhancing the NIR power and extending the welding duration resulted in denser and stronger joints. Under the radiation of NIR laser with maximum power of 0.5 W, the temperature of the hydrogel ANF-Fe₃O₄-PVA-2 raised to 470° C. in 5 seconds as recorded by an infrared thermal imaging system (FIG. 13). This temperature rise could be utilized to weld separate hydrogel strips and achieve high healing efficiency (FIG. 14 and FIG. 15). The rapid temperature elevation could be ascribed to the high particle density of Fe₃O₄—NH₂ nanoparticles on the surface of nanofibers, which could be further confirmed by numerical simulation on an array of nanoparticles (FIG. 16). The strategy of numerical simulation was as follows:

COMSOL Multiphysics (version 5.4a) was used to simulate the temperature on and around light-heated particles. Seven closely attached Fe₃O₄—NH₂ nanoparticles with a diameter of 25 nm were placed in a 1000 nm×1000 nm×1000 nm box filled with hydrogel as media. The model involves modules of heat transfer in solids.

$$∇ \cdot q = Q \qquad ( 1 )$$

$$q = - k ∇ T \qquad ( 2 )$$

where q was the heat flux vector, Q was heating source power, T was temperature, and k was thermal conductivity. The upper half surface of spherical particles was endowed with boundary heat source, which mimics light heating.

$$- n \cdot q = {Q}_{b} \qquad ( 3 )$$

The n was boundary normal vector. The boundary source power, Q_{b}, was set as 1.810 Wm⁻². The sides of the box were set to be at room temperature, 293.15 K. Due to the high-water content, the hydrogel was regarded to have same heat transfer properties as deionized water, possessing a thermal conductivity, 0.59 Wm⁻¹K⁻¹, while the thermal conductivity of Fe₃O₄—NH₂ nanoparticles was set as 5.9 Wm⁻¹K⁻¹. After these settings, the model simulated the temperature distribution on and around particles, where it was found the central part of the nanoparticle array to be more heated.

Referring to FIG. 16, the center of the array displayed a higher temperature than the end part under the illumination of the NIR laser, due to coupling of Fe₃O₄—NH₂ nanoparticles. Moreover, the internal heating source dispersed within the hydrogel matrix was beneficial for the high temperature rise since the aramid-based materials had a lower thermal conductivity.

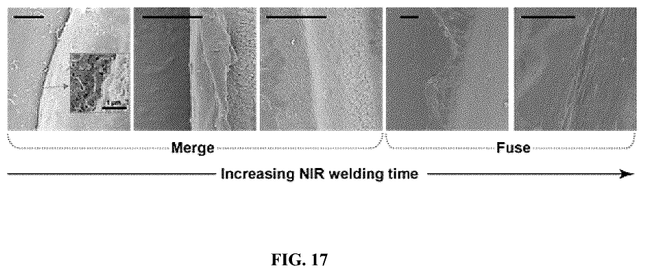

Investigations on the microscale morphology revealed the healing/welding process (FIG. 17). Under the NIR illumination, the elevated temperature enhanced the fluidity of the fibrous nanocomposites, providing driving forces that bypass the kinetic traps and reduce the free interfacial energy. The nanofibers connected and merged as they were softened by elevated temperature. The water-rich protective PVA layer mitigated the high transient temperature that might damage the fibers and provide time window for operation.

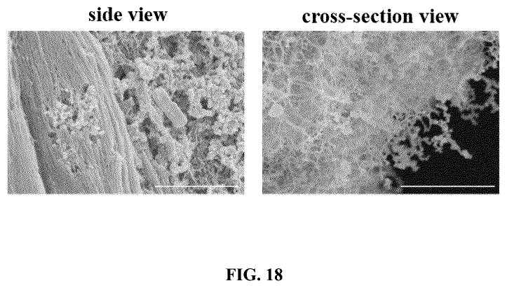

Further indicated by the change in external appearance of as-welded samples, the welding process resulted in densification of the networks. For thin film samples with 80 μm in thickness, the welded region became more transparent, while the same regions for thick stripes (250 μm in thickness) exhibited obvious shrink (FIG. 14). As a comparison, ANF-Fe₃O₄ aerogels were easily burned by NIR laser, even in the low laser intensity (FIG. 18). By increasing the illumination time, two hydrogels were fused together, of which the interface became smooth but condensed.

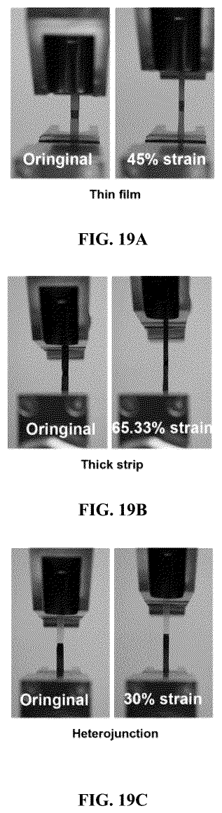

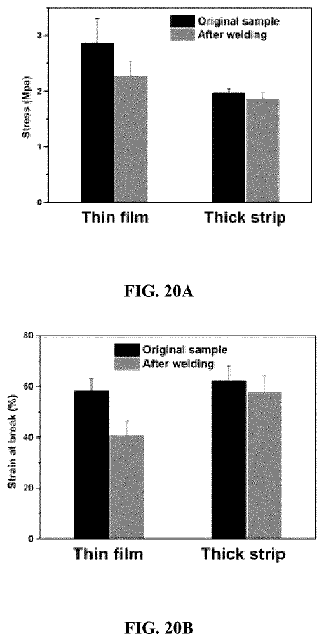

The mechanical properties of healed hydrogels were dependent on the thickness of samples, as indicated by tensile tests (FIG. 19A-19B). Besides the homogenous welding of the same type of hydrogels, the present method also realized “macroscopic assembly” by welding the ANF-Fe₃O₄-PVA and ANF-PVA hydrogels, forming heterojunctions, as indicated by their distinct colors (FIG. 7 and FIG. 19C). Tensile stress-strain curves indicated 65.33% fracture strain is retained by the healed hydrogel sample, achieving a high healing efficiency of at least 98%, when comparing to the 66.60% fracture strain of the original hydrogel sample (FIG. 15). The heterojunction resisted up to 42.73% strain and 0.8385 MPa stress before fracture. Nonuniform nanoparticle distribution might cause insufficient heating at the interface, resulting in reduced healing efficiency. Compared with healed stripe samples that retained 94.8% tensile modulus and 98.09% fracture strain, thin films samples exhibited a significantly decreased healing efficiency in modulus (79.4%) and strain (69.94%) (FIG. 20A-20B). Since the healing process was protected by PVA and interstitial water, under the same healing time, thinner films may experience more damage due to lower water content. To optimize the healing efficiency, finding the balance between water content, nanoparticle concentration, and welding power would worth future investigations.

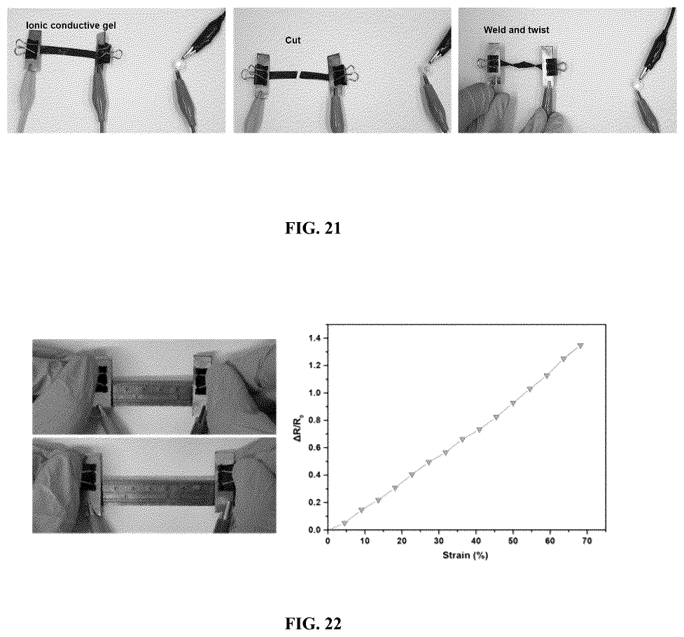

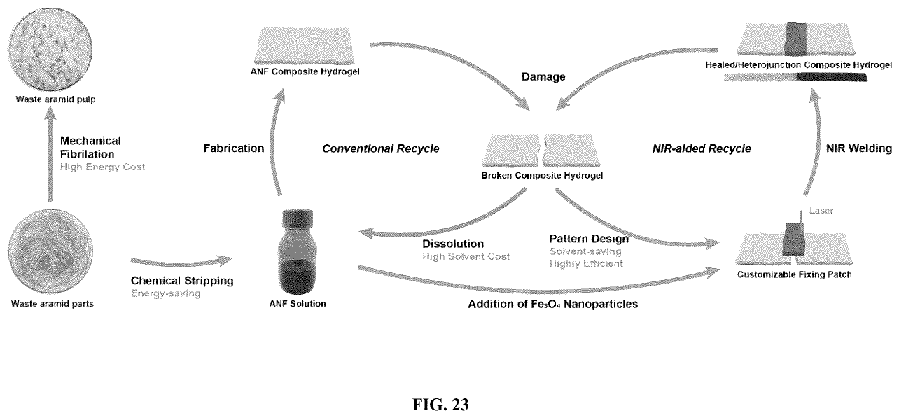

In addition, the hydrogel was employed as soft ionic conductors with infiltrated sodium citrate. This conductive hydrogel was also healable using NIR induced welding, and electrical conductivity of the hydrogel was maintained even under bending and twisting (FIG. 21). In addition to Fe₃O₄—NH₂ nanoparticles, the same fabrication method was used to make ANF-Au-PVA composite hydrogel (FIG. 22), as gold nanoparticles were biocompatible and exhibit good electrical conductivity. The resistance changes against strain exhibited a linear and monotonic increase when stretched up to 70%, the rupture strain of ANF-Au-PVA hydrogel. With gauge factor of 1.979, calculated by the resistance change divided by the strain, this linear trend showed a feasible electrical property as a strain sensor.

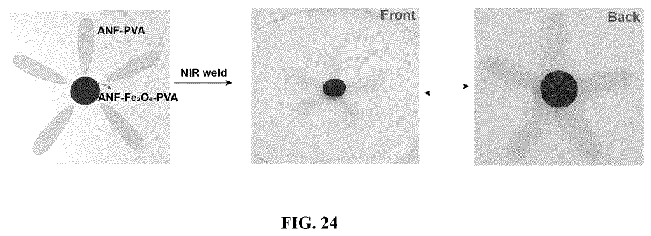

This unique approach to welding and healing aramid-based materials opens a new way for the improvement of device fabrication and materials recycling (FIG. 23). First, the traditional method to recycle the aramid materials faces severe challenges, including high energy/solvent cost, harsh degradation conditions, and, specifically for aramid composites, expensive purification process. The present method provides a new idea to recycle ANFs forming desirable shapes with high energy efficiency and without solvent aided physical and/or chemical treatment. Second, the ANFs have long been used as building blocks constructing highly connective networks as reinforcing components. It is not an easy task to dial in functionalities to such materials in a clickable manner or fabricate hybrid structures. The strategy of heterogenous welding in the present invention provides a customizable, modular approach that integrates various components with distinct functionalities.

### Example 5

Soft programmable actuators with the ANF-Fe₃O₄-PVA hydrogel

Due to the inherent magnetic nature of Fe₃O₄—NH₂ nanoparticles, the ANF-Fe₃O₄-PVA hydrogel can serve as programmable soft magnetic actuators. Existing magnetic hydrogels usually achieve actuation patterns based on anisotropic assembly of magnetic nanoparticles with predesigned alignments. However, the sophisticated assembly process may impose restrictions on device fabrication or mechanical performance.

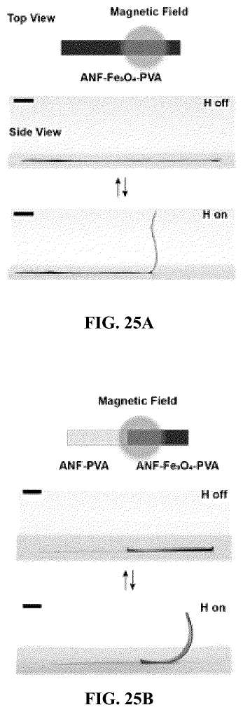

For ANF-Fe₃O₄-PVA hydrogels, due to their photothermal weldability, they could be integrated with non-magnetic ANF-PVA hydrogels and achieved modulated, controllable actuation patterns through hybrid structures (FIG. 24).

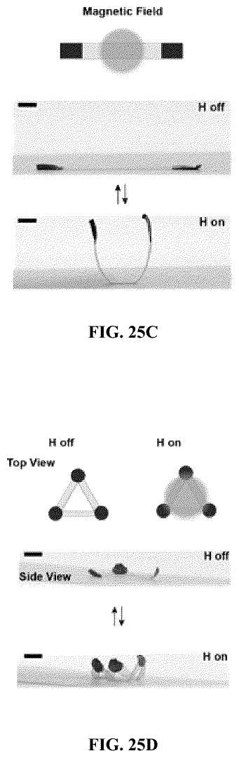

Actuation experiments were performed under an inhomogeneous 300 mT magnetic field perpendicular to the operation plane under ambient conditions. The pure ANF-Fe₃O₄-PVA long stripe demonstrated a bending motion under magnetic field adapting on one end (FIG. 25A). To further characterize the modulated functionality of the hydrogel samples, the anisotropic response of four hydrogel heterojunction samples under the magnetic field applied at the central position (FIGS. 25B-25D) were tested. Through different welding patterns, the actuators showed different transformations, demonstrating the possibility of programmable and customizable control of the magnetic actuation. The working principles also allowed for the construction of more complex shapes and patterns. For example, a flower-shaped hydrogel was formed by ANF-Fe₃O₄-PVA petals and an ANF-PVA core, potentially serving as a reversible robotic hand for grasping objects (FIG. 25E).

### Example 6

Cell culture and fluorescence characterization

The chemical stability of the hydrogels further limits hazardous waste production during polymer reconfiguration and shape morphing via photothermal effects, mechanical forces, and magnetic fields, ensuring a high degree of safety. This high stability of the hydrogels yields excellent biocompatibility even suitable for primary human cells such as chondrocytes.

First, the hydrogel samples were cut and washed with ethanol and phosphate buffered saline (Gibco™ pH 7.4 basic (1×)) prior to cell culture. Dulbecco's modified eagle medium (DMEM, Gibco™, high glucose), fetal bovine serum (Gibco™, qualified, Brazil), and Penicillin-Streptomycin (Gibco™, 10,000 U/mL) were mixed as received at 89%, 10%, 1% in volume fraction, respectively.

To further investigate the cytotoxicity and biocompatibility of the composite hydrogel, mouse fibroblast cell line NIH/3T3 was cultured on the ANF-Fe₃O₄-PVA hydrogel with three different particle concentrations (7.3 wt %, 13.7 wt %, and 24.1 wt %) without surface modification. The samples were characterized on day 1, 3, and 5 during culturing. To characterize the samples, LIVE/DEAD™ Cell Imaging Kit (488/570) (Invitrogen™) were used as received, the cells were treated for 15 min under ambient room conditions. The florescence was characterized using Nikon Eclipse Ci-L.

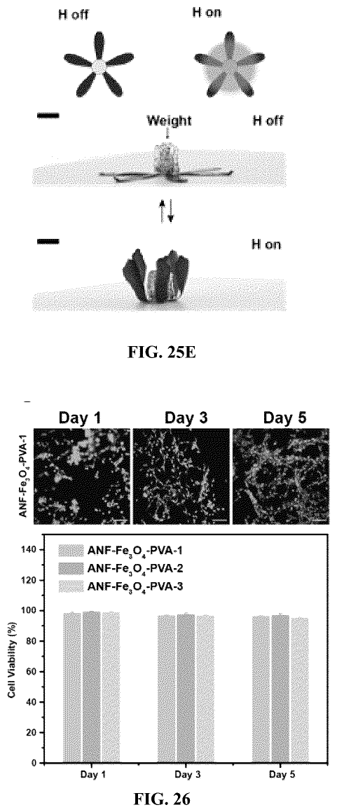

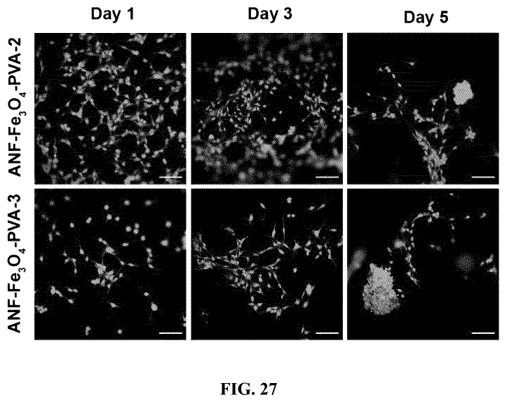

From day 1 to day 5, no obvious difference was observed in cell viability (FIG. 26 and FIG. 27).

In conclusion, the present invention introduces a facile and scalable approach to the fabrication of mechanically robust and magnetically active hydrogels with potential applications in soft robots and remotely controllable biomedical actuators. Fe₃O₄ nanoparticles functionalized with alkyl chains terminated with amine groups exhibit good stability in suspension while enabling desired interactions with ANFs. Stepwise assembly of ANFs, functionalized Fe₃O₄ nanoparticles, and polyvinyl alcohol (PVA) via solution-based mixing processes lead to magnetically responsive nanofiber network with excellent structural robustness and uniformity. In addition, the photothermal property of functionalized Fe₃O₄ nanoparticles and the reconfigurable hydrogen bonding within the nanofiber hydrogels affords fast and reliable welding with near infrared (NIR) radiation. NIR-laser-based photothermal welding enables production of Janus-type hydrogel structures or other complex and heterogeneous patterns, which allows for various modes of magnetic actuation with custom designs. The nanofibrous magnetic hydrogels also exhibit good biocompatibility, indicating potential applications in bio-integrated device systems.

By using NIR welding methods, the present invention achieves custom designed heterogenous structures with possibility of complex modes of deformation. It has demonstrated the ability to integrate various microelectronic sensors and electroactive polymers onto ANF-PVA based materials. In combination with the soft electronics, the multifunctional hydrogel platform may achieve more sophisticated sensing and actuation capabilities in a closed-loop manner. The hybrid device platform could enable diverse applications in implantable surgical tools, human-robot interactions, controlled release of drugs, and many other technologies.

### Definitions

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein and not otherwise defined, the terms “substantially,” “substantial,” “approximately” and “about” are used to describe and account for small variations. When used in conjunction with an event or circumstance, the terms can encompass instances in which the event or circumstance occurs precisely as well as instances in which the event or circumstance occurs to a close approximation. For example, when used in conjunction with a numerical value, the terms can encompass a range of variation of less than or equal to +10% of that numerical value, such as less than or equal to +5%, less than or equal to +4%, less than or equal to +3%, less than or equal to +2%, less than or equal to +1%, less than or equal to +0.5%, less than or equal to +0.1%, or less than or equal to +0.05%.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

### INDUSTRIAL APPLICABILITY

The present invention provides a new type of high-strength magnetic hydrogels, the materials therein combine tissue compliant mechanical strength, tissue similar water content, and photo-weldability with high efficiency via a simple and stepwise fabrication method.

ANF-based magnetic hydrogels have potential applications in various fields, including biomedical engineering, drug delivery systems, and tissue engineering, where their magnetic responsiveness and mechanical properties can be leveraged for specific functionalities. ADDIN EN.REFLIST

1. A magnetic hydrogel with a ternary magnetic composite, comprising:
a plurality of aramid nanofibers;
one or more magnetic components capable of dispersing in an organic solvent for at least 24 hours, wherein the organic solvent contains at least one hydrogen bonding acceptor; and
a polymer matrix,
wherein hydroxyl groups and amine groups on the one or more magnetic components spontaneous interact with the plurality of aramid nanofibers via hydrogen bonding, and the one or more magnetic components are embedded within the three-dimensional fibrous network formed by the plurality of aramid nanofibers.

2. The magnetic hydrogel of claim 1, wherein the plurality of aramid nanofibers comprises an aramid polymer.

3. The magnetic hydrogel of claim 1, wherein the one or more magnetic components comprise Fe₃O₄—NH₂ nanoparticles or gold nanoparticles.

4. The magnetic hydrogel of claim 1, wherein the one or more magnetic components have a particle size ranging from 15 nm to 50 nm.

5. The magnetic hydrogel of claim 1, wherein the concentration of the one or more magnetic components ranges from 5 wt % to 50 wt %.

6. The magnetic hydrogel of claim 5, when the concentration of the one or more magnetic components reaches to at least 20%, the magnetic hydrogel exhibits a similar mechanical robustness compared to a binary magnetic composite.

7. The magnetic hydrogel of claim 1, wherein the polymer matrix comprises polyvinyl alcohol and polyethylene glycol.

8. The magnetic hydrogel of claim 1, wherein the organic solvent comprises dimethyl sulfoxide, dimethylformamide, acetone, or a combination thereof.

9. The magnetic hydrogel of claim 1, wherein the magnetic hydrogel is further processed by near-infrared induced welding with one or more non-magnetic hydrogel to foam a heterogeneous hydrogel structure.

10. A method for fabricating a magnetic hydrogel, comprising the following steps:
mixing a colloidal suspension and an aramid nanofiber suspension in a solvent to form a first mixture, wherein the colloidal suspension comprises one or more magnetic components;
adding 2 mL to 10 mL of polyvinyl alcohol solution in the solvent to the first mixture to obtain a ternary mixture, wherein the ternary mixture is in a form of a viscous and moldable fluid without gelation; and
immersing the ternary mixture in water to form the magnetic hydrogel.

11. The method of claim 10, wherein step of immersing the ternary mixture in water to form the magnetic hydrogel further comprises performing solvent exchange.

12. The method of claim 10, wherein the plurality of aramid nanofibers comprises an aramid polymer.

13. The method of claim 10, wherein the one or more magnetic components comprise Fe₃O₄—NH₂ nanoparticles or gold nanoparticles, with a particle size ranging from 15 nm to 50 nm.

14. The method of claim 10, wherein the solvent comprises dimethyl sulfoxide, dimethylformamide, acetone, or a combination thereof.

15. The method of claim 10, wherein the ratio between the colloidal suspension and the aramid nanofiber suspension is 1:20 to 1:1.

16. The method of claim 10, wherein the ratio between the first mixture and the polyvinyl alcohol solution is 1:10 to 1:2.
